(19) United States
(12) Patent Application Publication
(10) Pub. No.: US 2022/0177406 A1
(43) Pub. Date: Jun. 9, 2022

(54) NOVEL PROCESS FOR PREPARATION OF KEY INTERMEDIATE OF PINOXADEN

(71) Applicant: BHAGIRADHA CHEMICALS & INDUSTRIES LIMITED, Hyderabad (IN)

(72) Inventors: S. CHANDRASEKHAR, Hyderabad (IN); B. PUNITH, Hyderabad (IN); B. SIVARAMI REDDY, Hyderabad (IN)

(73) Assignee: BHAGIRADHA CHEMICALS & INDUSTRIES LIMITED, Hyderabad (IN)

(21) Appl. No.: 17/600,959

(22) PCT Filed: Mar. 28, 2020
(86) PCT No.: PCT/IB2020/052976

§ 371 Date: Oct. 1, 2021

(30) Foreign Application Priority Data
Apr. 1, 2019 (IN) 201941013136

(51) Int. Cl. C07C 67/317; C07C 67/307

(52) U.S. Cl. CPC C07C 67/317; C07C 67/307

(57) ABSTRACT

A novel process for preparation of compounds of formula (I) used in the synthesis of Pinoxaden. The intermediate is a substituted phenyl malonic esters of formula (I), where R is C₁-C₄ alkyl, straight or branched chain.

### SUMMARY OF INVENTION

The present invention relates to a novel process for preparation of compounds of formula (I) used in the synthesis of Pinoxaden. The intermediate is a substituted phenyl malonic esters of formula (I), where R is C₁-C₄ alkyl, straight or branched chain.

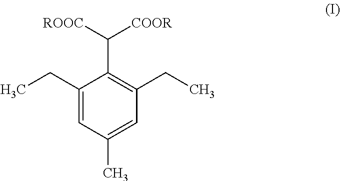

The synthesis process consists of the following steps:
A) Reacting Grignard salts of formula II with oxomalonic esters of formula III to form compound of formula IV
B) Halogenation of compound of formula IV to obtain compound of formula V
C) Hydrodehalogenation of compound of formula V to obtain compound of formula I.

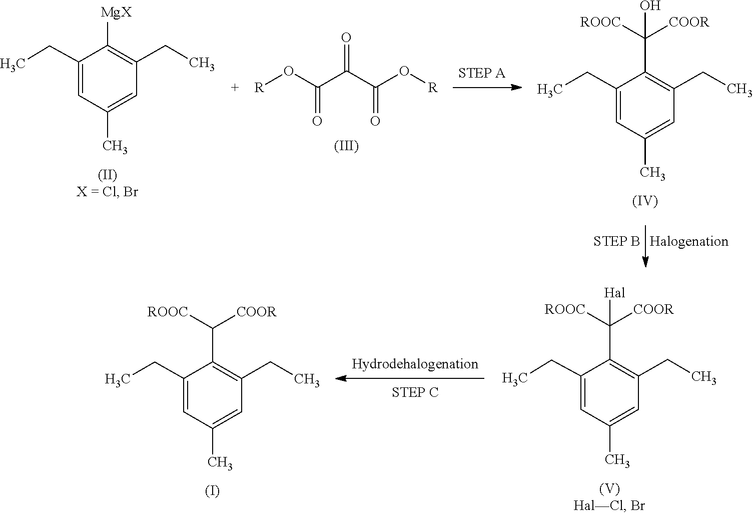

### DESCRIPTION AND BACKGROUND

Pinoxaden is a herbicide introduced by Syngenta which is used for control of weeds in Cereals. Compound of formula (I) is a key intermediate in synthesis of Pinoxaden. Compounds of formula (I) is reacted with mono or di salts of 1,4,5-oxadiazepane of formula (VI) in presence of acid binding agent such as triethylamine resulting in the formations of an intermediate of formula (VII). Compound of formula (VII) is further reacted with pivaloyl chloride resulting in the formation of Pinoxaden.

U.S. Pat. No. 6,552,187 describes the method of preparation of Pinoxaden starting from compounds of formula (I).

U.S. Pat. No. 7,268,247 describes the method of preparation of arylmalonic acid dinitriles by C—C linking of aryl halides with malononitrile. Preparation of compound of formula (I) from arylmalonic acid dinitriles are described in U.S. Pat. No. 6,552,187. The hydrolysis of nitrile to amide is done in concentrated sulfuric acid in a controlled manner in limited amount of water. The product is then extracted into solvent and spent acid is disposed off. Disposal of sulfuric acid is a major bottleneck in scale up and industrial application of the above process.

US patent application US 2009/0131708 A1 describes the method of producing substituted phenyl malonic esters by reacting Grignard salts of corresponding aryl bromides with oxomalonic acid esters. However it is carried out at very low temperatures preferably in the range of −80 deg C. to −40 deg C.

The present invention is a novel and simple process of producing substituted phenyl malonic esters of formula (I), where R is C₁-C₄ alkyl, straight or branched chain, which avoids use of sulfuric acid and low reaction temperatures. The synthesis consists of following steps
A) Reacting Grignard salts of formula II with oxomalonic acid esters of formula III, where R is straight or branched chain, to form compound of formula IV.
B) Halogenation of compound of formula IV with a halogenating agent to obtain compound of formula V
C) Hydrodehalogenation of compound of formula V to obtain compound of formula I.

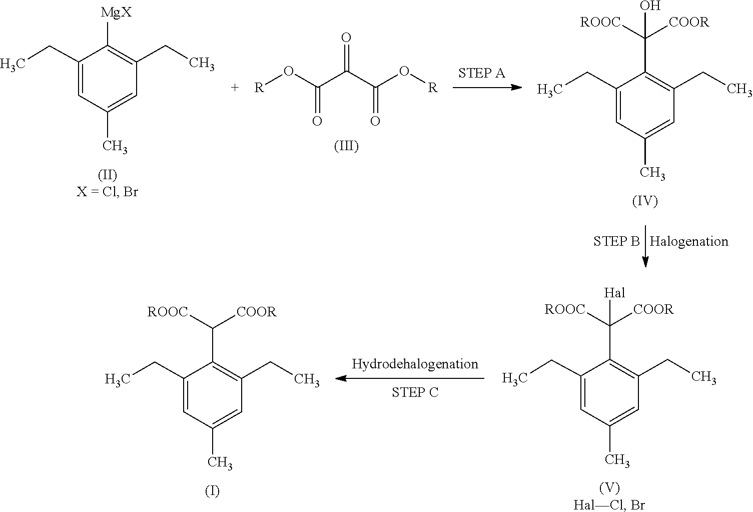

### DETAILED DESCRIPTION

The present invention relates to the process of preparation of substituted phenyl malonic esters of formula (I), where R is C₁-C₄ alkyl, straight or branched chain, which is a key intermediate in the synthesis of Pinoxaden.

Grignard salts of formula II can be prepared from corresponding aryl haldides of formula IIa, where halide is preferably a bromide.

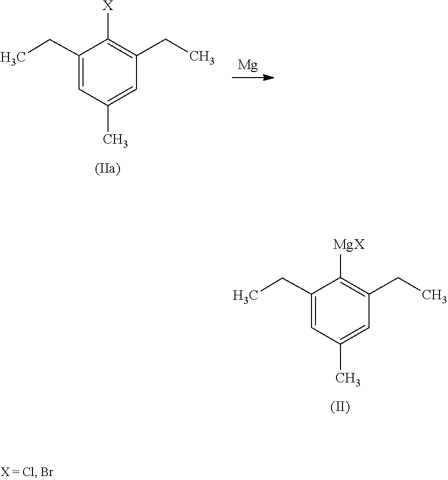

The reaction of arylbromide is carried out in Tetrahydrofuran (THF) at temperature between 0 and 100 deg C., preferably at room temperature. Ethylbromide is used as Grignard initiator. Dialkyloxomalonates can be prepared in a manner which is known from the literature (J. Org. Chem., 1981, 46, 2598). Compound of formula IV is formed by the reaction of Grignard salt with Dialkyloxomalonates. The reaction is carried out in THF at temperature between −80 to +30 deg C., preferably between −30 to 10 deg C.

Compound of formula IV which formed from the reaction of Grignard salt with oxomalonates, is further halogenated in suitable solvent such as chlorobenzene, toluene, dichloromethane, dichloroethane etc with a suitable halogenating agent like POCl₃, PCI₅, POBr₃, PBr₅, SOCl₂, in particular SOCl₂ to obtain compound of formula V. The reaction may be conducted at temperature between −20 deg C. to +40 deg C., preferably between −10 to +10 deg C.

Hydrodehalogenation of compound of formula V in carried out in suitable solvents like THF, toluene, xylene, alcohols such as methanol, ethanol, n-propanol, isopropanol, n-butanol, t-butanol, more preferably in methanol and ethanol to obtain compound of formula I. The reaction is preferably carried out in presence of catalyst selected from a group consisting of Platinum, Palladium, Nickel or Copper, preferably Pd/C, Pt/C, Raney-Ni. The reaction is carried out at temperature between 0 to 100 deg C., preferably between 35 to 70 deg C. The reaction is carried under hydrogen pressure between 1 kg/cm2 g to 20 kg/cm2 g, preferably between 2 to 10 kg/cm2 g.

Below are some examples for best understanding the process.

### Example

### Preparation diethyl-2-(2,6-diethyl 4-methyl phenyl) malonate

### Step A: Preparation of diethyl 2-hydroxy-2-(2,6-diethyl-4-methylphenyl)malonate

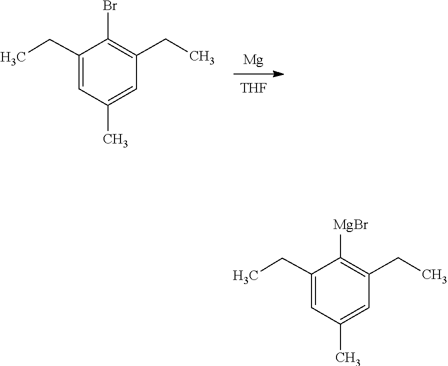

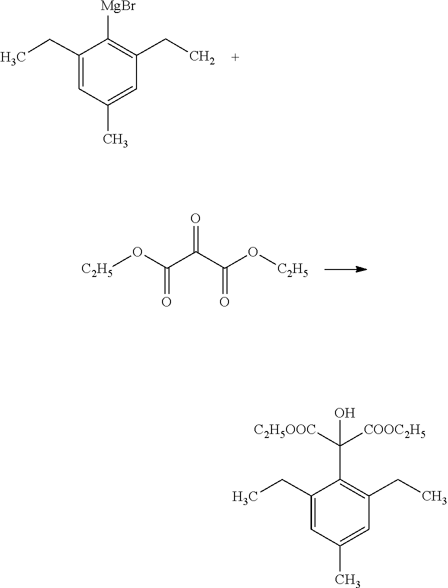

In a 2 liter 4-neck round bottom flask equipped with stirrer, thermometer, heater/cooling thermostat, nitrogen purging, charge 600 ml of tetrahydrofuran, 20 gm of magnesium turnings, 0.5 gm of ethyl bromide at room temperature. Add 106 gm of 2,6-diethyl-4-methyl bromobenzene at 35-40 deg C. over a period of 2 hr and maintain the reaction mixture till end of completion of 2,6-diethyl-4-methyl bromobenzene on GC. Separate the excess magnesium turnings from the reaction mass by decantation. Cool the reaction mixture to −15 deg C. and add 308 gm of diethyl oxomalonate in 2 hr while maintain the temperature of reaction mass. Maintain the reaction at −15 deg C. for 6 hr. Add 53 gm of 1N hydrochloric acid at 0 deg C. and stir the reaction mass for 60 min and separate the aqueous layer. Distill the solvent and excess oxomalonate from the organic layer under vacuum to obtain residue of 150 gm containing 125.1 gm of product corresponding to a yield of 84%.

### Step B: Preparation of diethyl 2-chloro-2-(2,6-diethyl-4-methylphenyl)malonate

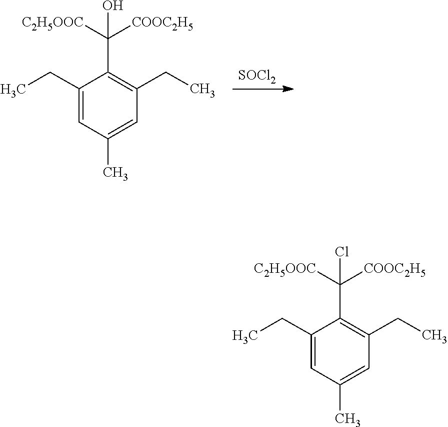

In a 2 liter 4-neck round bottom flask equipped with stirrer, thermometer, heater/cooling thermostat with nitrogen purging, charge 150 gm of diethyl 2-hydroxy-2-(2,6-diethyl-4-methylphenyl)malonate crude obtained from the previous step. Add 900 gm of dichloromethane and cool to 0 deg C. Add 35 gm of dimethyl formamide followed by 63 gm of thionyl chloride while maintaining the reaction mass at 0 deg C. Maintain the reaction mass till completion of starting material. At the end of reaction wash the organic layer with 2×400 ml of water and recover the solvent under rotavapor resulting in residue of 141 gm containing 125.8 gm of product corresponding to yield of 95%.

### Step C: Preparation of diethyl-2-(2,6-diethyl-4-methylphenyl)malonate

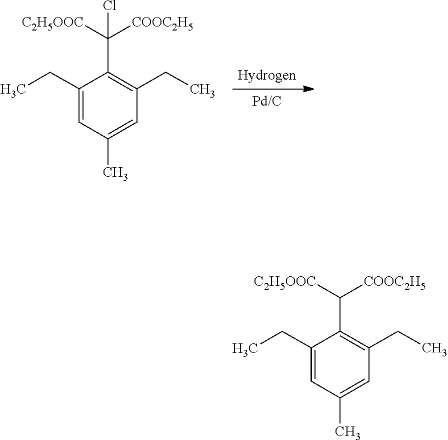

In a 2 liter autoclave charge 127 gm of diethyl 2-chloro-2-(2,6-diethyl 4-methylphenyl)malonate, 800 gm ethanol, 44 gm of triethylamine and 1 gm 5% palladium carbon at room temperature. Heat the reaction mass to 50 deg C. and maintain the autoclave at 5 kg/cm2 g hydrogen pressure till completion of the reaction. Cool to room temperature and filter to separate palladium carbon. Remove ethyl alcohol in a rotavapour and charge 500 gm of xylene. Filter triethylamine hydrochloride salt. Organic layer contains 107 gm of diethyl-2-(2,6-diethyl 4-methyl phenyl) malonate corresponding to yield of 95% of theory.

### Preparatory Example: Preparation of 8-(2,6-diethyl 4-methyl phenyl)-tetrahydro-pyrazolo[1,2-d][1,4,5]oxadiazepine-7,9-dione

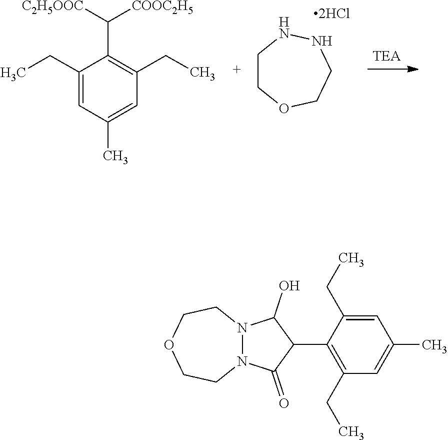

In a 2 liter 4-neck round bottom flask equipped with stirrer, thermometer, heater/cooling thermostat with nitrogen purging, charge xylene layer from previous contains 107 gm of diethyl (2,6-diethyl 4-methyl phenyl) malonate, 67 gm of 1,4,5-oxadiazepane dihydrochloride at room temperature. Then add 78 gm of triethylamine and heat to reflux, maintain the reaction at reflux temperature for 6 hr, cool to room temperature and filter the triethylamine hydrochloride. Xylene is recovered and the product is purified by re-precipitation of its sodium salt with aq HCl and dried to obtain 102 gm of white powder, yield 90% and purity is 98%.

### Preparatory Example: Preparation of Pinoxaden

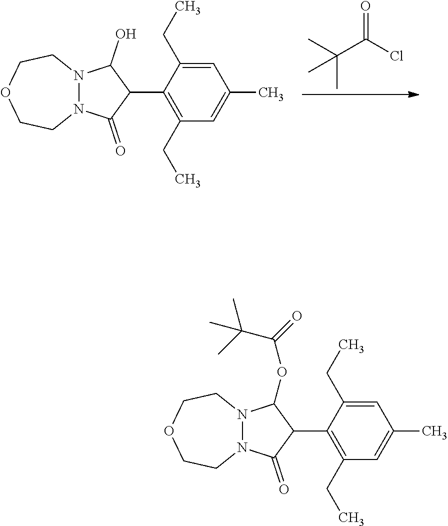

In a 2 liter 4-neck round bottom flask equipped with stirrer, thermometer, heater/cooling thermostat with nitrogen purging charge tetrahydrofuran 600 ml, 100 gm of 8-(2,6-diethyl 4-methyl phenyl)-tetrahydro-pyrazolo[1,2-d][1,4,5]oxadiazepine-7,9-dione and cool to 20° C. and charge 67 gm of triethyl amine and 1 gm of 4-dimethyl amino pyridine and add 50 gm pivaloyl chloride. Maintain the reaction at 20-25° C. till completion of the reaction and wash the reaction mass with 20% brine solution. Organic layer is concentrated and the product is recrystallized in methyl tertbutyl ether and dried to obtain 97 gm of Pinoxaden of purity 98.1% corresponding to yield 75%.

1. A process for preparation of compound of formula-I, through novel route comprising the steps of:
A. Reacting Grignard salts of formula Il with oxomalanoic acid esters of formula Ill, where R is C1-C4 alkyl, straight or branched chain, to form compound of formula IV.
B. Halogenation of compound of formula IV in a solvent along with halogenating agent to obtain compound of formula V
C. Hydrodehalogenation of compound of formula V to obtain compound of formula I. 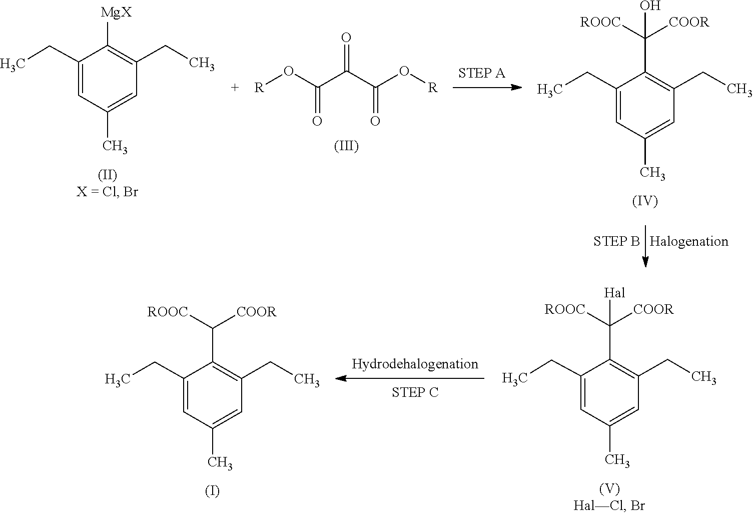
wherein Formula-1, R is C₁-C₄ alkyl, straight or branched chain.

2. The process of claim 1, where in step A is carried out in tetrahydrofuran solvent at temperatures between −30° C. to +10° C.

3. The process of claim 1, where in step B is carried out between −10° C. to +10° C.

4. The process of claim 1, where in step B is carried out in one of a solvent such as Chlorobenzene, Toluene, Dichloromethane, Dichloroethane.

5. The process of claim 1, where in halogenating agent in step B is POCl₃, PCl₅, POBr₃, PBr₅, or SOCl₂

6. The process of claim 1, where in step C is carried out in presence of catalyst selected from a group consisting of Platinum, Palladium, Nickel or Copper.

7. The process of claim 1, where in step C is carried out in one of the alcohols based solvent such as methanol, ethanol, n-propanol, isopropanol, n-butanol, and t-butanol.

8. The process of claim 1, where in step C is carried out at temperature between 35° C. to 70° C.

9. The Process of claim 1, where Step C is carried under hydrogen pressure between 2 to 10 kg/cm2 g.
